(19) United States
(12) Plant Patent Application Publication
(10) Pub. No.: US 2004/0083522 P1
(43) Pub. Date: Apr. 29, 2004

(54) Kalanchoe plant named 'Patty'

(75) Inventor: A. Smaal, (US)

Correspondence Address: WOOD, PHILLIPS, KATZ, CLARK & MORTIMER, 500 West Madison Street, Suite 3800, Chicago, IL, 60661-2511, US

(73) Assignee: A.B. BREEDING BV

(21) Appl. No.: 10/644,507

(22) Filed: Aug. 20, 2003

Related U.S. Application Data
(63) Continuation of application No. 09/993,406, filed on Nov. 19, 2001, now abandoned.

(51) Int. Cl. A01H 5/00

(52) U.S. Cl. PLT/340

(57) ABSTRACT

A new and distinct cultivar of Kalanchoe plant named ‘Patty’ is provided. This new cultivar is characterized by its bright orange flowers, dark green foliage, compact growth habit and excellent basal branching.

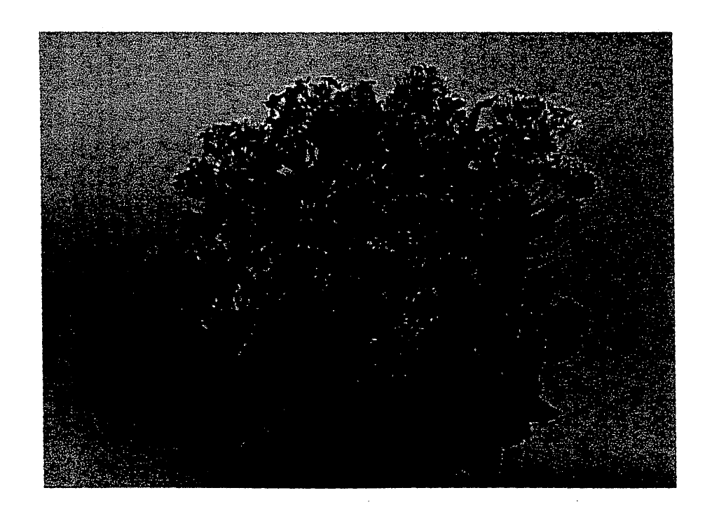

### GENUS AND SPECIES OF PLANT CLAIMED

Kalanchoe blossfeldiana

### VARIETY DENOMINATION

Patty

### BACKGROUND OF THE INVENTION

The present invention comprises a new and distinct Kalanchoe plant, botanically known as Kalanchoe blossfeldiana, and hereinafter referred to by the cultivar name ‘Patty’.

The new cultivar was developed by the inventor through a controlled breeding program during August 1995 in The Netherlands. The female (seed) parent of ‘Patty’ was the proprietary Kalanchoe blossfeldiana selection designated 95013 (not patented), characterized by its small orange flowers and good branching habit. The male (pollen) parent of ‘Patty’ was the proprietary Kalanchoe blossfeldiana selection designated 95316-16 (not patented), characterized by its red flowers and dark green foliage. ‘Patty’ was discovered and selected as a flowering plant within the progeny of the stated cross by the inventor in June 1996.

Asexual reproduction of the new cultivar by terminal cuttings has demonstrated that the characteristics of the new cultivar as herein described are firmly fixed and retained through successive generations of such asexual propagation.

### SUMMARY OF THE INVENTION

It was found that the cultivar of the present invention:

(a) exhibits bright orange flowers,

(b) forms dark green foliage,

(c) exhibits a good basal branching character and

(d) exhibits a compact upright growth habit.

The new cultivar of the present invention can be compared to ‘Arjuno’ (U.S. Plant Pat. No. 6,632). In side by side comparisons with ‘Arjuno’, it is found that the new cultivar is more compact, less floriferous and exhibits flowers of a different color. A detailed comparison is found in Table A.

### BRIEF DESCRIPTION OF PHOTOGRAPH

The accompanying photograph shows as nearly true as it is reasonably possible to make the same in color illustrations of this type, typical flower and foliage characteristics of the new cultivar. The plants were grown in a greenhouse at West Chicago, Ill., U.S.A.

### DETAILED BOTANICAL DESCRIPTION

The cultivar ‘Patty’ has not been observed under all possible environmental conditions to date. Accordingly, it is possible that the phenotype may vary somewhat with variations in the environment, such as temperature, light intensity, and day length.

The chart used in the identification of colors described herein is the R.H.S. Colour Chart of The Royal Horticultural Society, London, England. The color values were determined on Jan. 4, 2001 between 9:00 and 9:30 am under natural daylight. The plants used for the following observations and measurements were produced from cuttings taken from stock plants and grown in West Chicago, Ill. in 10 cm pots for nineteen (19) weeks. Plants were grown in a double poly carbonate covered greenhouse under conditions comparable to those used in commercial practice while utilizing a soilless growth medium and maintaining temperatures of approximately 82° F. during the day and approximately 66° F. during the night. Plants were exposed to three weeks of long days followed by 9 weeks of short days. Growth regulators were applied as follows: B nine, 3000 ppm at days 17 and 28 and 1500 ppm day 42.

Classification:

Botanical.—Kalanchoe blossfeldiana, cultivar Patty.

Parantage:

Male (pollen) parent.—Proprietary Kalanchoe blossfeldiana selection #95316-16.

Female (seed) parent.—Proprietary Kalanchoe blossfeldiana selection #95013.

Propagation:

Type cutting.—Terminal tip.

Time to initiate roots.—Approximately 7-14 days with the shorter times generally being experienced in the summer and the longer times in the winter.

Time to develop roots.—Approximately 21 days.

Root description.—Fibrous, branching.

Plant description:

Habit of growth.—Moderately vigorous with good basal branching. A mature plant, at 16 weeks after the planting of an unrooted cutting, commonly measures approximately 13.6 cm in height and approximately 17.1 cm in width.

Branching habit.—Typically forms one main stem 10 cm in length and 4 mm in diameter and 14 lateral branches approximately 7.5 cm in length and 4 mm in diameter.

Plant form.—Upright and mounded.

Foliage.—Form: Simple. Arrangement: Opposite. Shape: Ovate with obtuse apex, cuneate base and crenate margin. Texture: Glabrous, succulent. Size of mature foliage: Approximately 8.7 cm in length and approximately 5.5 cm in width. Color of mature foliage: Upper surface closest to 137A, lower surface closest to 147B. Petiole length: Approximately 1 cm. Petiole color 137C.

Flower description:

Flowering habit.—Freely flowering.

Natural flowering season.—Early January. Flowering time is dependent on temperature, light intensity and other growing conditions.

Flowering response time.—Approximately 10 weeks.

Inflorescence type.—Dichasial cymes.

Flowers borne.—Above foliage.

Flower longevity.—Approximately 7 days.

Fragrance.—None.

Quantity of flowers.—Approximately 37 flowers on main stem and 24 flowers on each lateral branch.

Flower buds.—Shape: Elliptical, becoming elongated as it matures. Size just before opening: Length is approximately 1.3 cm, width is approximately 3 mm. Color: 34C.

Flower.—Salverform Corolla: Diameter is approximately 1.6 cm, depth is approximately 4 mm and aspect is slightly cupped. Corolla tube is 9 mm in length, 3 mm in diameter.

Petals.—Number: Four. Shape: Obovate with cuspidate apex, rounded base and entire margin. Approximate size: 8 mm in length, 5 mm in width. Aspect: Slightly convex. Texture: Glabrous and velvety.

Sepals.—Number: Four. Shape: Lanceolate with acuminate apex and entire margin. Size: 8 mm in length, 1.5 mm in width. Texture: Glabrous. Color: Upper and lower surface: Between 144B and 144C.

Mature flower color.—Upper surface of petals is closest to 39A fading to 45C at senescence; lower surface of petals is closest to 31D.

Peduncle.—Length: Approximately 1.3 cm in length and 2 mm in diameter.

Pedicel length.—Approximately 7 mm.

Reproductive organs.—Androecium: 8 stamens arranged in two groups of four—one group being shorter than the other. Anther shape is oblong and flat, length is approximately 0.5 mm. Pollen color is 3B. Gynoecium: Stigma shape is flat, appearance is crystalline, colorless and transparent. Style: Length is approximately 2 mm, color is 154B. Ovary: 4 celled, 7 mm in length, color is 144B.

Seed development: Seed development has not been observed.

Disease resistance: Resistance to known Kalanchoe pathogens has not been observed.

I claim:

1. A new and distinct cultivar of Kalanchoe plant named ‘Patty’ substantially as herein shown and described, which:
(a) exhibits bright orange flowers,
(b) forms dark green foliage,
(c) exhibits a good basal branching character and
(d) exhibits a compact upright growth habit.
